(19) United States
(12) Patent Application Publication
(10) Pub. No.: US 2022/0015685 A1
(43) Pub. Date: Jan. 20, 2022

(54) SYSTEMS AND METHODS FOR BRAIN STATE CAPTURE AND REFERENCING

(71) Applicant: Dhiraj JEYANANDARAJAN, Irvine, CA (US)

(72) Inventor: Dhiraj JEYANANDARAJAN, Irvine, CA (US)

(21) Appl. No.: 16/928,321

(22) Filed: Jul. 14, 2020

(51) Int. Cl. A61B 5/00; A61B 5/0482; A61B 5/04; A61B 5/117

(52) U.S. Cl. CPC A61B 5/4064; A61B 5/0482; A61B 5/7405; A61B 5/7267; A61B 5/7455; A61B 5/04012; A61B 5/117; A61B 5/742

(57) ABSTRACT

Systems and methods for brain state capture and referencing are provided. In some embodiments, the method comprises receiving first raw electroencephalograph (EEG) data of a user from at least one EEG sensor; processing the first raw EEG data of the user; generating a current brain state based on the processed first raw EEG data; receiving a selection of a target brain state, wherein the target brain state was previously generated by processing second raw EEG data; responsive to receiving the selection, generating a comparison of the current brain state and the target brain state; and presenting an output to the user based on the comparison, wherein the output comprises at least one of: a visual output; an aural output; and a tactile output.

### FIELD OF THE DISCLOSURE

This disclosure relates to systems and methods for detection, capture, decomposition, and use of brain state activity based on a user-selected state and subsequent notification to the user about whether their then current state is or is not consistent with the user-selected state.

### SUMMARY

In general, one aspect disclosed features a system, comprising: a hardware processor; and a non-transitory machine-readable storage medium encoded with instructions executable by the hardware processor to perform a method comprising: receiving first raw electroencephalograph (EEG) data of a user from at least one EEG sensor; processing the first raw EEG data of the user; generating a current brain state based on the processed first raw EEG data; receiving a selection of a target brain state, wherein the target brain state was previously generated by processing second raw EEG data; responsive to receiving the selection, generating a comparison of the current brain state and the target brain state; and presenting an output to the user based on the comparison, wherein the output comprises at least one of: a visual output; an aural output; and a tactile output.

Embodiments of the system may include one or more of the following features. In some embodiments, the second raw EEG is received from at least one of: the user; and one or more other users. In some embodiments, the output represents at least one difference between the current brain state and the target brain state. In some embodiments, the method further comprises: generating the target brain state, comprising: receiving second raw EEG data, processing the second raw EEG data, and creating the target brain state based on the processed second raw EEG data. In some embodiments, the method further comprises: creating a record of the target brain state based on the target brain state, an identity of one or more users from whom the second raw EEG data is received, and a location of the EEG sensor that collected the raw EEG data. In some embodiments, the method further comprises: training a machine learning model with the target brain state; wherein comparing the current brain state with the target brain state comprises: providing the current brain state as an input to the trained machine learning model, wherein the trained machine learning model outputs the comparison based on the current brain state. In some embodiments, processing the first raw EEG data of the user comprises: isolating at least one spectral brain wave component of the first raw EEG data of the user.

In general, one aspect disclosed features a non-transitory machine-readable storage medium encoded with instructions executable by a hardware processor of a computing component, the machine-readable storage medium comprising instructions to cause the hardware processor to perform a method comprising: receiving first raw electroencephalograph (EEG) data of a user from at least one EEG sensor; processing the first raw EEG data of the user; generating a current brain state based on the processed first raw EEG data; receiving a selection of a target brain state, wherein the target brain state was previously generated by processing second raw EEG data; responsive to receiving the selection, generating a comparison of the current brain state and the target brain state; and presenting an output to the user based on the comparison, wherein the output comprises at least one of: a visual output; an aural output; and a tactile output.

Embodiments of the non-transitory machine-readable storage medium may include one or more of the following features. In some embodiments, the second raw EEG is received from at least one of: the user; and one or more other users. In some embodiments, the output represents at least one difference between the current brain state and the target brain state. In some embodiments, the method further comprises: generating the target brain state, comprising: receiving second raw EEG data, processing the second raw EEG data, and creating the target brain state based on the processed second raw EEG data. In some embodiments, the method further comprises: creating a record of the target brain state based on the target brain state, an identity of one or more users from whom the second raw EEG data is received, and a location of the EEG sensor that collected the raw EEG data. In some embodiments, the method further comprises: training a machine learning model with the target brain state; wherein comparing the current brain state with the target brain state comprises: providing the current brain state as an input to the trained machine learning model, wherein the trained machine learning model outputs the comparison based on the current brain state. In some embodiments, processing the first raw EEG data of the user comprises: isolating at least one spectral brain wave component of the first raw EEG data of the user.

In general, one aspect disclosed features a method, comprising: receiving first raw electroencephalograph (EEG) data of a user from at least one EEG sensor; processing the first raw EEG data of the user; generating a current brain state based on the processed first raw EEG data; receiving a selection of a target brain state, wherein the target brain state was previously generated by processing second raw EEG data; responsive to receiving the selection, generating a comparison of the current brain state and the target brain state; and presenting an output to the user based on the comparison, wherein the output comprises at least one of: a visual output; an aural output; and a tactile output.

Embodiments of the method may include one or more of the following features. In some embodiments, the second raw EEG is received from at least one of: the user; and one or more other users. In some embodiments, the output represents at least one difference between the current brain state and the target brain state. Some embodiments comprise generating the target brain state, comprising: receiving second raw EEG data, processing the second raw EEG data, and creating the target brain state based on the processed second raw EEG data. Some embodiments comprise creating a record of the target brain state based on the target brain state, an identity of one or more users from whom the second raw EEG data is received, and a location of the EEG sensor that collected the raw EEG data. Some embodiments comprise training a machine learning model with the target brain state; wherein comparing the current brain state with the target brain state comprises: providing the current brain state as an input to the trained machine learning model, wherein the trained machine learning model outputs the comparison based on the current brain state.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 depicts a block diagram of an example of a brainwave system according to embodiments of the disclosed technology.

FIG. 2 illustrates a brainwave referencing process according to embodiments of the disclosed technology.

FIG. 3 illustrates a process for creating reference brain states according to embodiments of the disclosed technology

FIG. 4 is an example computing component that may be used to implement various features of embodiments described in the present disclosure.

The figures are not exhaustive and do not limit the present disclosure to the precise form disclosed.

### DETAILED DESCRIPTION

Brainwaves may be detected via electroencephalography (EEG), which may involve monitoring and recording electrical impulse activity of the brain, typically noninvasively. For example, EEG data can be generated by placing a number of electrodes (often part of an EEG test headset) on or near the subject's scalp. The electrodes may detect the electrical impulses generated by the brain and send signals to a computer that may record the results. The data from each electrode may be deemed a channel representing data from a portion of the brain where the electrode is located. Each channel may have a reference electrode in a montage used in differential amplification of the source signal.

Brainwaves may be detected as a time-varying signal, and may comprise components having different spectral characteristics such as frequency, power, and the like. As an example, brainwaves may include the following brainwave components: delta waves, theta waves, alpha waves, beta waves, and gamma waves. The spectral characteristics of the brainwaves may indicate different brain states based on factors such as source location, duration, coherence, and dominance or amplitude.

EEG data has been used primarily for medical or research purposes, but with consumer grade recording devices becoming more prevalent there are increasing uses for the general population.

Computer processing allows the analysis of spectral characteristics into component parts that may be used to categorize brainwaves during a specific activity (e.g., training or other specific activities). Conventional approaches involve the acquisition of baselines. These baselines involve typical, known actions that elicit a predictable change in the brainwave signals. Typically, the brain state is captured over a continuous period and/or during predetermined activity or set of activities.

In embodiments of the disclosed technology, an EEG headset may capture the spectral characteristics of a user's brainwaves as a snapshot of brain activity at a user-selected time (e.g., when a user is in a particular brain state). The system may be trained through the capture of a single or multiple instances of a specific brain state in order to achieve a high specificity for a particular user's spectral characteristics during a particular brain state.

When an EEG headset is worn at other times, the stored spectral characteristics of a user's brain states may be compared to the current brain state and the system may provide a prompt to the user to bring awareness to their current brain state in comparison to the stored brain state that they may be targeting to operate within. Visual, auditory or tactile prompts may be used to bring a user's awareness to their current brain state as it deviates from the target state.

Various embodiments of the present disclosure may include systems and methods for training a computer system (e.g., via machine learning) on the biometric data corresponding to a particular target state for a user. According to one aspect of the invention, the system may be configured to receive raw EEG data generated from a single multi-channel EEG headset (or other device) connected to a user. Alternatively the system may be configured to receive raw EEG data generated from multiple multi-channel EEG headsets (or other devices) that are connected to multiple users. An isolation component may be configured to run the EEG data through various levels of signal processing to isolate components from the spectral characteristics of the EEG signal. By way of example the raw EEG from each channel may be run through a fast Fourier transform (FFT) to separate out various frequency components in each channel, isolating the brainwave components (e.g., alpha, beta, theta, delta, gamma components) for each channel for pattern classification. In some implementations, the EEG data may be run through a high and low bandpass filter prior to the filtered data being run through the FFT to isolate the spectral frequencies of each channel. A user interface may be provided that allows a user or third parties to indicate either a subjective or objective state when a desired brain state is achieved. By way of example a user may indicate a highly focused state, a peaceful state, or a relaxed state that they would like stored. A time window around the user indicated state is created and the spectral data is stored. The stored state may be labeled in that moment or at a future time. Multiple instances may be captured at subsequent times for the same state.

This initial capture of the spectral characteristics of specific brain states constitutes a training period. The training period may involve the analysis and characterization of the spectral features of the captured data that is stored under the particular labeled brain state category. In some embodiments, a machine learning model may be trained during this period. This capture may be triggered based on a user input that indicates that the user would like to capture their then current brain state.

In subsequent uses of the EEG headset, the user may indicate a preset target brain state category that the user would like to achieve. The isolated spectral characteristics of the live biometric signals are compared to the set of previously captured and categorized targeted brain state. The system may alert the user to their brain state status relative to the learned target brain state with visual, auditory, or tactile indicators, and the like.

In various implementations, a computer system may selectively consider two or more of the isolated components of the separated brainwaves from each channel and/or region based on, for example, location of source/destination, frequency, timing, and/or mental state. For example, the processing component may utilize ratios and/or other statistical methods to simultaneously consider multiple isolated components. Certain internal physical sources of brainwaves may be associated with a specific action or thought process so that considering multiple components (e.g. alpha and gamma) from that source produces a clearer signal representing the thought process being performed. Certain brainwave components may also indicate mood or mental states. Thus, brainwave components from several sources may be simultaneously considered or evaluated (e.g., by determining ratios between separate components).

Group dynamics combining the large scale dynamics of brainwave components across two or more users may be considered. For example, a group of users working on a specific project may wear EEG headsets. The system may take the collective input from these multiple sources and combine the information for a group state characterization to indicate the highest level of productivity within the group. The system may improve accuracy based on machine learning algorithms for the user and for population comparison across common brain states.

These and other features and characteristics of the system and/or method disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

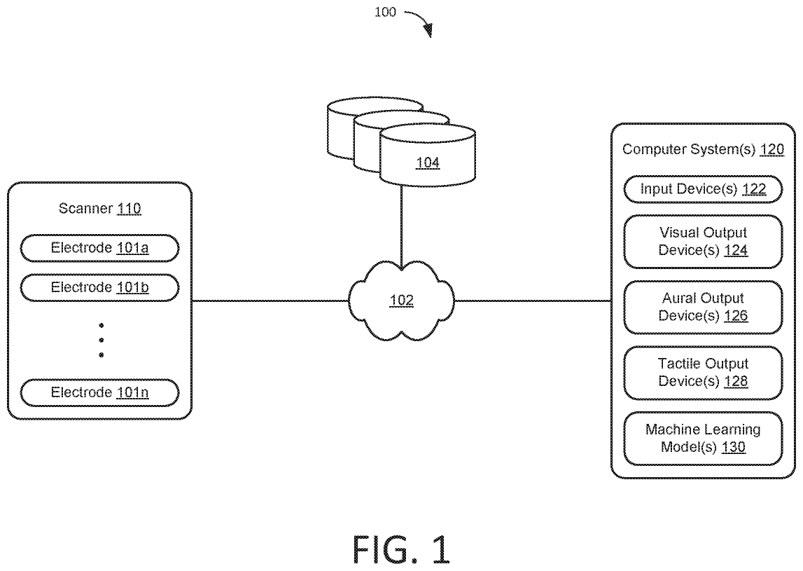

FIG. 1 depicts a block diagram of an example brainwave system 100 according to embodiments of the disclosed technology. In various implementations, the system 100 may include one or more storage devices 104, a scanner 110, a computer systems 120, and/or other components. In various implementations, the scanner 110 may comprise a head-mounted device and/or other device. In various implementations, the scanner 110 may comprise an electroencephalography (EEG) device configured to sense the electrical activity inside a person's brain. For example, the scanner 110 may comprise a device that connects to the scalp of a patient at multiple points via electrodes 101, or connects directly to the brain or skull via inserted probes as in electrocorticography (ECoG), intracranial electroencephalography (iEEG), or subdural EEG (SD-EEG). In various implementations, the scanner 110 may comprise a multi-channel EEG headset.

In various implementations, the scanner 110 may include one or more electrodes 101 (e.g., electrode 101a and electrodes 101b through 101n). The scanner 110 may comprise anywhere from a low density (e.g., 2-channel system) to a high density (e.g., 256-channel system) array of electrodes. In various implementations, each electrode 101 may be attached to a patient's head (or scalp) and configured to receive brainwaves. For example, the scanner 110 may comprise a 4-channel EEG system with a ground and reference. In some implementations, each electrode 101 may correspond to a specific channel input of the scanner. For example, an electrode 101a may correspond to a channel 101a, an electrode 101b may correspond to a channel 101b, etc.

The channels of each electrode may be configured to receive delta, theta, alpha, beta, and/or gamma signals—each of which may correspond to a given frequency range. In a non-limiting example implementation, delta waves may correspond to signals between 0 and 3.5 Hz, theta waves may correspond to signals between 3.5 and 8 Hz, alpha waves may correspond to signals between 8 and 12 Hz, beta waves may correspond to signals between 12 and 30 Hz, and gamma waves may correspond to signals above 30 Hz. These example frequency ranges are not intended to be limiting and are to be considered exemplary only.

In some implementations, the electrodes 101 may be attached at locations spread out across the patient's head (or scalp) and/or centered over each of the primary regions of the brain. The electrodes 101 may be configured to detect electric potentials in the brain from the low ionic current given off by the firing of synapses and neural impulses traveling within neurons in the brain. These electric potentials may repeat or be synchronized at different frequencies according to the previously listed brainwave types (e.g. alpha and beta). These frequency ranges may be separated from the single superimposed frequency signal detected at each electrode by scanner 110 or computer systems 120, as described further herein. In various implementations, this isolation, separation, decomposition, or deconstruction of the signal is performed via application of an FFT.

In various implementations, the computer systems 120 may be configured to receive raw EEG data generated by the scanner 110. In some implementations, the scanner 110 and/or the computer systems 120 may be configured to perform initial signal processing on the detected brainwaves. For example, the scanner 110 and/or the computer systems 120 may be configured to run the raw EEG data through high and low bandpass filters prior to the filtered data being run through an FFT to isolate the spectral frequencies of each channel. For example, each channel may be run through a high and low bandpass filter. In some implementations, the scanner 110 and/or the computer systems 120 may be configured to perform error detection, correction, signal decomposition, signal recombination, and other signal analysis. Accordingly, one or both of the scanner 110 and the computer systems 120 may be configured to filter, analyze, and/or otherwise process the signals captured by the scanner 110.

In an example implementation using the 10-20 international system of electrode placement, Channel 1 may correspond to the Fp1 location, Channel 2 may correspond to Fp2, Channel 3 may correspond to T5, and Channel 4 may correspond to T6. The ground and reference electrodes may be placed on either side of the earlobe. As described herein, filtered data for each channel may be run through an FFT to isolate the spectral frequencies of each channel. The power of the theta (e.g., 4-7 Hz), alpha (e.g., 8-12 Hz), beta (e.g., 13-20 Hz), and gamma (e.g., 21-50 Hz) components of each channel for a given sampled timeframe (e.g., 3 seconds) may be determined. The power of each of the isolated components may be used to generate a visualization of the brainwave components, for example as described below.

In various implementations, the computer systems 120 may output data to be displayed to a patient, physician, and/or other user via one or more visual output devices 124 as described further herein. In some implementations, the computer systems 120 may be connected to one or more cloud servers and/or storage devices configured to store the EEG data and visualizations generated by the computer systems 120. Accordingly, the visualizations may be retrieved and viewed at a later time. For example, one or more cloud servers and/or associated storage may be implemented as an electronic health records (EHR) database. In various implementations, the computer systems 120 may be configured to provide processing capability and perform one or more operations as described herein.

The computer system(s) 120 may include one or more computers. The computers may be of various types, including general-purpose and special-purpose computers. The computer system(s) 120 may include one or more input devices 122, for example including keyboards, mice, pointers, touchscreens, and the like. The computer system(s) 120 may include one or more visual output devices 124, for example including display panels, touchscreens, traffic light style indicators, and the like. The computer system(s) 120 may include one or more aural output devices 126, for example such as loudspeakers, headphones, and the like. The computer system(s) 120 may include one or more tactile output devices 128, for example such as vibration devices, electrical stimulation devices, and the like. The computer system(s) 120 may include one or more machine learning models 130, which may be trained by supervised methods, unsupervised methods, other methods, or combinations thereof. The elements of the system 100 may be interconnected by a network 102, by direct links, or by combinations thereof. The network 102 may include the Internet.

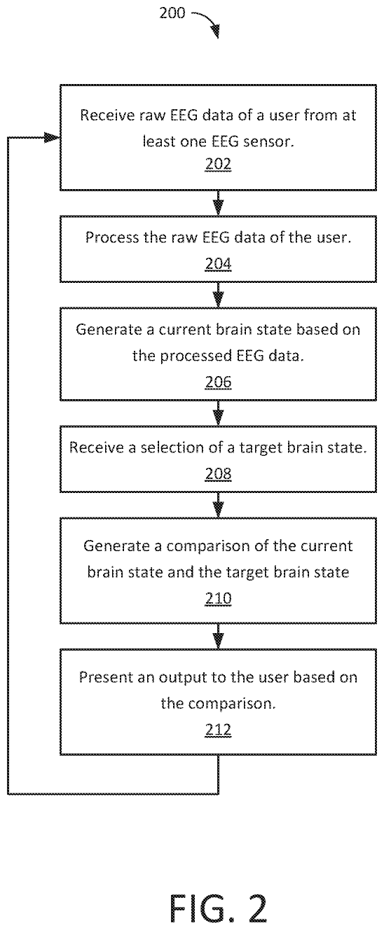

FIG. 2 illustrates a brainwave referencing process 200 according to embodiments of the disclosed technology. Although the elements of the processes disclosed herein are presented in a particular order, it should be understood that in various embodiments one or more elements may be performed in a different order, in parallel, or omitted. The process 200 may be implemented, for example, in the brainwave system 100 of FIG. 1. In embodiments of the process 200, a user employs a brainwave system to select a target brain state, and to guide the user to achieve that target brain state.

Referring to FIG. 2, the process 200 may include receiving raw EEG data of the user from at least one EEG sensor, at 202. In the example of FIG. 1, the raw EEG data may be generated in real time by a scanner 110 having one or more electrodes 101. For example, the scanner 110 may be implemented as an EEG headset, or like. The raw EEG data may be received from the scanner 110 by the computer systems 120 of FIG. 1.

Referring again to FIG. 2, the process 200 may include processing the raw EEG data of the user, at 204. In the example of FIG. 1, the raw EEG data received from scanner 110 may be processed by the computer systems 120, for example as described above. For example, the raw EEG data may be filtered and then processed to isolate its spectral components, for example using one or more filters and an FFT process.

Referring again to FIG. 2, the process 200 may include generating a current brain state based on the processed EEG data, at 206. As used herein, the term “brain state” may refer to a collection of parameters characterizing components of the brainwaves of one or more users. The parameters characterizing components of the brainwaves may include, for example, frequencies, amplitudes, durations, and the like. In the example system 100 of FIG. 1, the current brain state may be generated by computer systems 120, and may include reference to stored in data storage devices 104. In some embodiments, the computer systems 120 may present a representation of the current brain state to the user using one or more of the output devices 124, 126, and 128. For example, the visual output devices 124 may present a graphical representation of the parameters characterizing components of the brain where this for example such as a bar chart, pie chart, real-time graph of the parameters over time, and the like. Additional visualizations may be generated as described in co-pending commonly-owned U.S. patent application No. (TBD—Attorney Docket No. 45WN-296421), the disclosure thereof incorporated by reference herein in its entirety for all purposes.

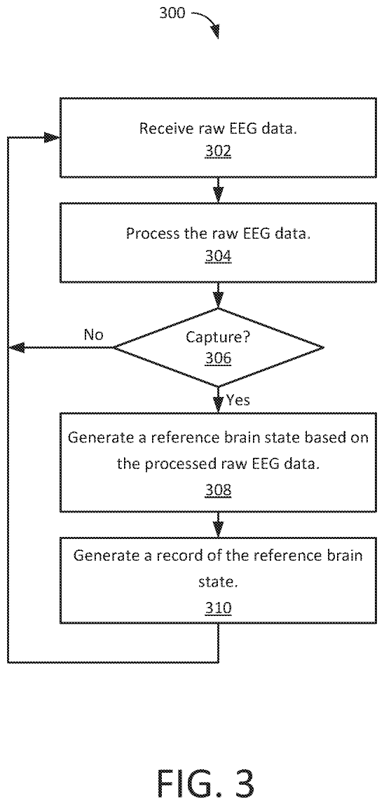

Referring again to FIG. 2, the process 200 may include receiving a selection of a target brain state, at 208. In the example system 100 of FIG. 1, the brainwave system 100 may store a number of previously-recorded reference brain states. The stored reference brain states may include brain states of the user, brain states one or more other users, or combinations thereof. Each stored reference brain state may be labeled, for example as “calm,” “focused,” “energetic,” and the like. The stored reference brain states may be generated and labeled as described in detail below with reference to FIG. 3.

The computer systems 120 may provide a graphical user interface that provides a catalog of the stored reference brain states. For example, the interface may list the stored reference brain states by label, category, user, date, and the like. The user may employ the graphical user interface to choose a target brain state that the user would like to achieve from among the stored reference brain states.

Referring again to FIG. 2, the process 200 may include generating a comparison of the current brain state and the target brain state responsive to receiving the user's selection of the target brain state, at 210. In the example system 100 of FIG. 1, the computer systems 120 may generate the comparison by comparing characteristics of corresponding components of the current brain state of the target brain state. For example, the computer systems 120 may compare parameters of corresponding brainwave spectral components of the current brain state and the target brain state. In some embodiments, the comparison may be performed wholly or in part using the machine learning models 130. For example, the machine learning models may be trained with processed EEG data of one or more users, which may include the current user, as described in detail below with reference to FIG. 3. In this example, the comparison may be generated by providing processed EEG data as an input to the trained machine learning models 130, which in response to this input provides the comparison as an output.

Referring again to FIG. 2, the process 200 may include presenting an output to the user based on the comparison, at 212. In the example system 100 of FIG. 1, the computer systems 120 may present the output using the visual, aural, and tactile output devices 124, 126, and 128. In some embodiments, the output may comprise representations of the current brain state and the target brain state. In some embodiments, the output may represent at least one difference between the current brain state of the user and the target brain state, thereby enabling the user to move toward the target brain state by reducing these differences. For example, the output may be a graphical display representing the differences between corresponding brainwave spectral components of the current brain state and the target brain state.

Referring again to FIG. 2, the process 200 may repeat as often as needed, thereby enabling the user to guide the user's brain state toward the target brain state.

FIG. 3 illustrates a process 300 for creating reference brain states according to embodiments of the disclosed technology. The process 300 may be implemented, for example, in the brainwave system 100 of FIG. 1.

Referring to FIG. 3, the process 300 may include receiving raw EEG data, at 302. In the example of FIG. 1, the raw EEG data may be generated in real time by a scanner 110 having one or more electrodes 101. For example, the scanner 110 may be implemented as an EEG headset, or like. The raw EEG data may be received from the scanner 110 by the computer systems 120 of FIG. 1. In some embodiments, the brain state may be captured while the user or users are performing a particular activity. In embodiments where the reference brain state is to represent a single user, the raw EEG data is collected only from the user. For example, upon attaining a desired brain state, the user may control the system to capture that brain state for future reference. In embodiments where the reference brain state to represent multiple users, the raw EEG data is collected from multiple users. In either of these embodiments, the raw EEG data for each brain state may be collected on a single occasion or on multiple occasions. In embodiments where the reference brain state is to represent multiple users, the raw EEG data may be collected from the multiple users concurrently or at different times.

Referring again to FIG. 3, the process 300 may include processing the raw EEG data of the user, at 304. In the example of FIG. 1, the raw EEG data may be processed by the computer systems 120, for example as described above. For example, the raw EEG data may be filtered and then processed to isolate its spectral components, for example using an FFT process. In embodiments where the reference brain state is to represent multiple users, the processed EEG data may be combined. For example, corresponding spectral components may be combined from multiple users perform a single set of spectral components. In this example, each combined component may be characterized by one or more parameters, for example as described above for a single user.

The reception and processing of the raw EEG data may continue until it is decided that a particular brain state has been achieved, and should be captured, at 306. The decision to capture a particular brain state may be made by the user, by a technician monitoring the process, automatically when certain conditions are met, or any combination thereof.

Referring again to FIG. 3, the process 300 may include generating a reference brain state based on the processed raw EEG data, at 308. The generated reference brain state may be a collection of parameters characterizing components of the brainwaves of one or more users. The parameters characterizing components of the brainwaves may include, for example, frequencies, amplitudes, durations, and the like. In the example system 100 of FIG. 1, the reference brain state may be generated by computer systems 120.

Referring again to FIG. 3, the process 300 may include generating and storing a record of the reference brain state, at 310. Each reference brain state record may include metadata. For example, the metadata may include data describing the user(s) from whom the brain state was generated, the type of scanner used to collect the raw EEG data, locations of sensors used to collect the raw EEG data, the date on which the reference brain state was generated, and the like. The metadata may also include a label for the reference brain state. The label may be provided by the user(s), by a technician monitoring the process, and the like, and may be provided when the reference brain state is captured, or at some later time. Referring again to FIG. 3, the process 300 may include storing the captured reference brain state. In the example of FIG. 1, the captured reference brain state may be stored as a database record in the storage devices 104.

In some embodiments, the reference brain state records may be used to train a machine learning model, for example such as the machine learning models 130 of the brainwave system 100 of FIG. 1. The information in the brain state record, including the metadata, may be provided to the machine learning model in a supervised training mode, in an unsupervised training mode, or a combination thereof. Once trained, the machine learning model may be used in the brainwave referencing process 200 of FIG. 2.

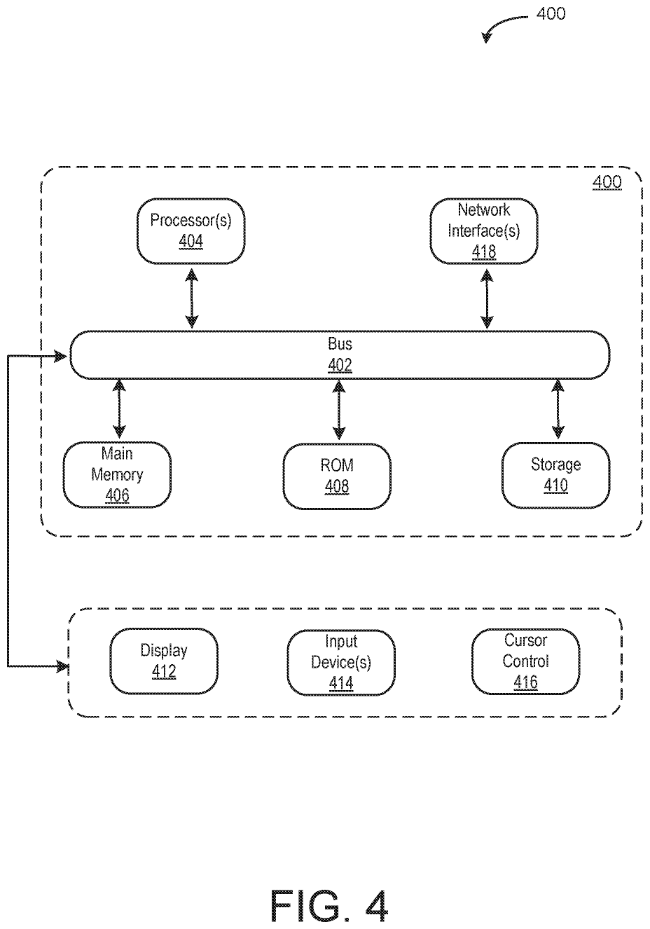

FIG. 4 depicts a block diagram of an example computer system 400 in which embodiments described herein may be implemented. The computer system 400 includes a bus 402 or other communication mechanism for communicating information, one or more hardware processors 404 coupled with bus 402 for processing information. Hardware processor(s) 404 may be, for example, one or more general purpose microprocessors.

The computer system 400 also includes a main memory 406, such as a random access memory (RAM), cache and/or other dynamic storage devices, coupled to bus 402 for storing information and instructions to be executed by processor 404. Main memory 406 also may be used for storing temporary variables or other intermediate information during execution of instructions to be executed by processor 404. Such instructions, when stored in storage media accessible to processor 404, render computer system 400 into a special-purpose machine that is customized to perform the operations specified in the instructions.

The computer system 400 further includes a read only memory (ROM) 408 or other static storage device coupled to bus 402 for storing static information and instructions for processor 404. A storage device 410, such as a magnetic disk, optical disk, or USB thumb drive (Flash drive), etc., is provided and coupled to bus 402 for storing information and instructions.

The computer system 400 may be coupled via bus 402 to a display 412, such as a liquid crystal display (LCD) (or touch screen), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. In some embodiments, the same direction information and command selections as cursor control may be implemented via receiving touches on a touch screen without a cursor.

The computing system 400 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “component,” “engine,” “system,” “database,” data store,” and the like, as used herein, can refer to logic embodied in hardware or firmware, or to a collection of software instructions, possibly having entry and exit points, written in a programming language, such as, for example, Java, C or C++. A software component may be compiled and linked into an executable program, installed in a dynamic link library, or may be written in an interpreted programming language such as, for example, BASIC, Perl, or Python. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

The computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor(s) 404 executing one or more sequences of one or more instructions contained in main memory 406. Such instructions may be read into main memory 406 from another storage medium, such as storage device 410. Execution of the sequences of instructions contained in main memory 406 causes processor(s) 404 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 410. Volatile media includes dynamic memory, such as main memory 406. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 402. Transmission media can also take the form of acoustic or light waves, such as those generated during radio-wave and infra-red data communications.

The computer system 400 also includes a communication interface 418 coupled to bus 402. Network interface 418 provides a two-way data communication coupling to one or more network links that are connected to one or more local networks. For example, communication interface 418 may be an integrated services digital network (ISDN) card, cable modem, satellite modem, or a modem to provide a data communication connection to a corresponding type of telephone line. As another example, network interface 418 may be a local area network (LAN) card to provide a data communication connection to a compatible LAN (or a WAN component to communicate with a WAN). Wireless links may also be implemented. In any such implementation, network interface 418 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet.” Local network and Internet both use electrical, electromagnetic or optical signals that carry digital data streams. The signals through the various networks and the signals on network link and through communication interface 418, which carry the digital data to and from computer system 400, are example forms of transmission media.

The computer system 400 can send messages and receive data, including program code, through the network(s), network link and communication interface 418. In the Internet example, a server might transmit a requested code for an application program through the Internet, the ISP, the local network and the communication interface 418.

The received code may be executed by processor 404 as it is received, and/or stored in storage device 410, or other non-volatile storage for later execution.

Each of the processes, methods, and algorithms described in the preceding sections may be embodied in, and fully or partially automated by, code components executed by one or more computer systems or computer processors comprising computer hardware. The one or more computer systems or computer processors may also operate to support performance of the relevant operations in a “cloud computing” environment or as a “software as a service” (SaaS). The processes and algorithms may be implemented partially or wholly in application-specific circuitry. The various features and processes described above may be used independently of one another, or may be combined in various ways. Different combinations and sub-combinations are intended to fall within the scope of this disclosure, and certain method or process blocks may be omitted in some implementations. The methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain of the operations or processes may be distributed among computer systems or computers processors, not only residing within a single machine, but deployed across a number of machines.

As used herein, a circuit might be implemented utilizing any form of hardware, or a combination of hardware and software. For example, one or more processors, controllers, ASICs, PLAs, PALs, CPLDs, FPGAs, logical components, software routines or other mechanisms might be implemented to make up a circuit. In implementation, the various circuits described herein might be implemented as discrete circuits or the functions and features described can be shared in part or in total among one or more circuits. Even though various features or elements of functionality may be individually described or claimed as separate circuits, these features and functionality can be shared among one or more common circuits, and such description shall not require or imply that separate circuits are required to implement such features or functionality. Where a circuit is implemented in whole or in part using software, such software can be implemented to operate with a computing or processing system capable of carrying out the functionality described with respect thereto, such as computer system 400.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

Although the present technology has been described in detail, for the purpose of illustration, based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system for generating an indication of a user's brain state relative to a stored, user selected brain state, the brain states being determined based on EEG data generated by an EEG sensor connected to the user, the system comprising:
a hardware processor; and
a non-transitory machine-readable storage medium encoded with instructions executable by the hardware processor, which when executed cause the system to be configured to:
store one or more previously-recorded brain states of the user;
generate a user interface to present options to the user to select at least one of the stored brain states as a target brain state;
receive a selection from the user of a target brain state;
receive first raw electroencephalograph (EEG) data of a user from at least one EEG sensor;
process the first raw EEG data of the user;
generate a current brain state based on the processed first raw EEG data;
compare the current brain state to the user selected target brain state; and
present an output to the user on an output device based on the comparison of the current brain state to the user selected target brain state, wherein the output comprises at least one of:
a visual output;
an aural output; and/or
a tactile output.

2. (canceled)

3. The system of claim 1, wherein the output represents at least one difference between the current brain state and the target brain state.

4. (canceled)

5. The system of claim 1, the system being further configured to:
store a target brain state along with an identity of one or more users from whom the second raw EEG data is received, and a location of the EEG sensor that collected the raw EEG data.

6. The system of claim 1, the system being further configured to:
train a machine learning model with the target brain state;
compare the current brain state with the target brain state based on the current brain state as an input to the trained machine learning model, and the trained machine learning model outputs the comparison based on the current brain state.

7. The system of claim 1, wherein processing the first raw EEG data of the user comprises:
isolating at least one spectral brain wave component of the first raw EEG data of the user.

8. A non-transitory machine-readable storage medium encoded with instructions executable by a hardware processor of a computing component for generating an indication of a user's brain state relative to a stored, user selected brain state, the brain states being determined based on EEG data generated by an EEG sensor connected to the user, the machine-readable storage medium comprising instructions to cause the hardware processor to be configured to:
store one or more previously-recorded brain states of the user;
generate a user interface to present options to the user to select at least one of the stored brain states as a target brain state;
receive a selection from the user of a target brain state;
receive first raw electroencephalograph (EEG) data of a user from at least one EEG sensor;
process the first raw EEG data of the user;
generate a current brain state based on the processed first raw EEG data;
compare the current brain state to the user selected target brain state; and
present an output to the user on an output device based on the comparison of the current brain state to the user selected target brain state, wherein the output comprises at least one of:
a visual output;
an aural output; and/or
a tactile output.

9. (canceled)

10. The non-transitory machine-readable storage medium of claim 8, wherein the output represents at least one difference between the current brain state and the target brain state.

11. (canceled)

12. The non-transitory machine-readable storage medium of claim 11, the system being further configured to:
store a target brain state along with an identity of one or more users from whom the second raw EEG data is received, and a location of the EEG sensor that collected the raw EEG data.

13. The non-transitory machine-readable storage medium of claim 8, the system being further configured to:
train a machine learning model with the target brain state;
compare the current brain state with the target brain state based on the current brain state as an input to the trained machine learning model, and the trained machine learning model outputs the comparison based on the current brain state.

14. The non-transitory machine-readable storage medium of claim 8, wherein processing the first raw EEG data of the user comprises:
isolating at least one spectral brain wave component of the first raw EEG data of the user.

15. A method for generating an indication of a user's brain state relative to a stored, user selected brain state, the brain states being determined based on EEG data generated by an EEG sensor connected to the user, the method comprising:
storing one or more previously-recorded brain states of the user;
generating a user interface to present options to the user to select at least one of the stored brain states as a target brain state;
receiving a selection from the user of a target brain state;
receiving first raw electroencephalograph (EEG) data of a user from at least one EEG sensor;
processing the first raw EEG data of the user;
generating a current brain state based on the processed first raw EEG data;
comparing the current brain state to the user selected target brain state; and
presenting an output to the user on an output device based on the comparison of the current brain state to the user selected target brain state, wherein the output comprises at least one of:
a visual output;
an aural output; and/or
a tactile output.

16. (canceled)

17. The method of claim 15, wherein the output represents at least one difference between the current brain state and the target brain state.

18. (canceled)

19. The method of claim 18, further comprising:
storing a record of the target brain state along with, an identity of one or more users from whom the second raw EEG data is received, and a location of the EEG sensor that collected the raw EEG data.

20. The method of claim 15, further comprising:
training a machine learning model with the target brain state;
comparing the current brain state with the target brain state based on the current brain state as an input to the trained machine learning model, and the trained machine learning model outputs the comparison based on the current brain state.

21. The system of claim 1, wherein the output comprises an alert when the current brain state corresponds to a user selected target brain state that the user would like to achieve.

22. The system of claim 1, wherein the system is configured to store the brain states with a label and the user interface is configured to present options for the user to select a stored brain state based on the labels associated with the stored brain states.

23. The system of claim 1, wherein the system is configured to capture a brain state at a particular time based on a user selection, store the captured brain state and associate and store a user selected label for the captured brain state.

24. The non-transitory machine-readable storage medium of claim 8, wherein the output comprises an alert when the current brain state corresponds to a user selected target brain state that the user would like to achieve.

25. The non-transitory machine-readable storage medium of claim 8, wherein the instructions cause the hardware processor to be further configured to store the brain states with a label and the user interface is configured to present options for the user to select a stored brain state based on the labels associated with the stored brain states.

26. The non-transitory machine-readable storage medium of claim 8, wherein the instructions cause the hardware processor to be further configured to capture a brain state at a particular time based on a user selection, store the captured brain state and associate and store a user selected label for the captured brain state.

27. The method of claim 15, wherein the output comprises an alert when the current brain state corresponds to a user selected target brain state that the user would like to achieve.

28. The method of claim 15, further comprising:
storing the brain states with a label and the user interface is configured to present options for the user to select a stored brain state based on the labels associated with the stored brain states.

29. The method of claim 15, further comprising:
capturing a brain state at a particular time based on a user selection, store the captured brain state and associate and store a user selected label for the captured brain state.
